(19) United States
(12) Patent Application Publication
(10) Pub. No.: US 2013/0142740 A1
(43) Pub. Date: Jun. 6, 2013

(54) SUNSCREEN COMPOSITIONS CONTAINING A NOVEL PRESERVATIVE SYSTEM

(71) Applicants: Paula CZIRYAK, Eatontown, NJ (US); Anil Shah, East Windsor, NJ (US); Ana Kljuic, New York, NY (US); Fabrice Laize, Paris (FR)

(72) Inventors: Paula CZIRYAK, Eatontown, NJ (US); Anil Shah, East Windsor, NJ (US); Ana Kljuic, New York, NY (US); Fabrice Laize, Paris (FR)

(73) Assignee: LOREAL S.A., Paris (FR)

(21) Appl. No.: 13/310,314

(22) Filed: Dec. 2, 2011

(51) Int. Cl. A61K 8/36; A61Q 17/04

(52) U.S. Cl. 424/60; 424/59

(57) ABSTRACT

The present invention is directed to a sunscreen composition containing a novel preservative system for inhibiting micro-organisms comprising (a) at least one aromatic carboxylic acid, or salt thereof; (b) from about 0.01% to about 1% by weight of at least one at least one aromatic alcohol; (c) at least one sunscreen active; and (d) a cosmetically acceptable carrier.

### FIELD OF THE INVENTION

The present invention is directed to sunscreen compositions containing a novel preservative system. More particularly, the invention is directed to a sunscreen composition containing a preservative system comprised of an aromatic carboxylic acid and aromatic alcohol that preserves sunscreen compositions as well as paraben preservatives.

### BACKGROUND OF THE INVENTION

Aging skin is the result of more than just chronological age. Skin is exposed to various environmental stresses, such as UV rays, which cause free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical which may attack adjacent fatty acids to generate new carbon radicals. This process can lead to a chain reaction producing lipid peroxidation products. Damage to the cell membrane can result in loss of cell permeability, increased intercellular ionic concentration and/or decreased ability to excrete or detoxify waste products. The end result is a loss of elasticity of the skin and the appearance of wrinkles leading to premature ageing of the skin. This process is commonly referred to as photo-aging. Compositions containing sunscreens are highly popular and may be used to prevent photo-aging.

Sunscreen products frequently require preservatives because of the high risk of spoiling due to mold, fungi and bacteria. Preservative ingredients having antimicrobial or antifungal activity are often added to sunscreen and cosmetic compositions to protect them from contamination, decay and/or spoilage. As a result, the products are protected from decomposition and deterioration and remain stable during the shelf life of the product. A disadvantage of using preservatives such as parabens, for examples, is that they may cause adverse effects such as irritation and allergic response.

It is an object of the present invention to provide sunscreen compositions with high PFA/SPF properties that do not require the use of parabens, yet are stable over long periods of time and yield a cosmetically elegant feel.

### BRIEF SUMMARY OF THE INVENTION

Disclosed herein is a preservative system for use in topical compositions which include sun care and sunscreen compositions. It has been found that a preservative system using from about 0.01% to about 1% of an aromatic alcohol in combination with an aromatic carboxylic acid results in a preservative system that is as effective as a paraben-containing preservative system for use in sunscreen compositions. Without being bound by theory, it is believed that the aromatic carboxylic acid, preferably p-anisic acid, interacts with the aromatic alcohol, preferably phenoxyethanol, to inhibit microorganisms in the composition. Advantageously, the combination effectively inhibits microorganism proliferation so that use of preservatives from the paraben class can be avoided. As a result, the composition provides a preservative system that does not require the use of parabens.

The present invention is directed to a sunscreen composition containing:

(a) at least one aromatic carboxylic acid, or salt thereof;
(b) from about 0.01% to about 1% by weight of at least one at least one aromatic alcohol;
(c) at least one sunscreen active; and
(d) a cosmetically acceptable aqueous carrier.

In yet another embodiment, the present invention is directed to a sunscreen composition containing:

(a) from about 0.01% to about 1% by weight of at least one aromatic carboxylic acid comprising an anisic acid, in the form of alkali or alkaline-earth metal salts, ammonium salts or salts with an organic amine, or one of its C₁₋₄ alkyl esters;
(b) from about 0.1% to about 1% by weight of at least one aromatic alcohol comprising phenoxyethanol;
(c) from about 1% to about 50% by weight of at least one sunscreen active; and
(d) remainder by weight to 100%, a cosmetically acceptable aqueous carrier.
wherein the weights of (a)-(d) are based on the total weight of the composition;
wherein the total amount of (a) and (b) is less than about 1.7%; and
wherein the composition has a pH ranging from about 4.0 to about 6.7.

The present invention is also directed to a method for protecting a keratinous substrate from harmful UV rays by topically applying the above-disclosed composition onto the keratinous substrate.

### DETAILED DESCRIPTION OF THE INVENTION

Other than in the operating examples, or where otherwise indicated, all numbers expressing quantities of ingredients and/or reaction conditions or ratios are to be understood as being modified in all instances by the term “about” which encompasses ±10%.

The present invention is based on the surprising and unexpected finding that the combination of about 0.01% to about 1% of an aromatic alcohol with an aromatic carboxylic acid inhibits microorganisms in cosmetic compositions, preferably sunscreen compositions. This inhibition of microorganisms results in a safe, stable product having long-term stability and thus improved photo protection efficacy, without needing parabens to be employed.

The present invention's composition surprisingly and unexpectedly results in the formation of a unique preservative system that prevents microbial growth and proliferation without the need for the use of paraben preservatives. Not only are paraben preservatives avoided, but the present invention's composition also yields a milder preservative system that mitigates adverse effects such as irritation and allergic responses.

A “physiologically acceptable medium” or “cosmetically acceptable medium” means a medium which is not toxic and can be applied to the skin, lips, hair, scalp, lashes, brows, nails or any other cutaneous region of the body. The composition of the invention may especially constitute a cosmetic or dermatological composition.

The subject composition can be provided in a plethora of forms, including but not limited to creams, liquid, gel, cream-gel, lotion, serum, a powder or a solid tube, and may optionally be packaged as an aerosol and may be in the form of a mousse or a spray, prepared according to the usual methods.

### Aromatic Carboxylic Acid

The sunscreen composition containing preservatives for inhibiting micro-organisms contains at least one aromatic carboxylic acid, or salt thereof.

Aromatic carboxylic acids or salts generally include those chosen from benzoic acid, para-anisic acid, anisic acid, caffeic acid, chlorogenic acid, diphenolic acid, ferulic acid, hippuric acid, hydroxycinnamic acid, phenylthioglycolic acid, salicylic acid, acetylsalicylic acid, phthalic acid, salified forms thereof, and combinations thereof.

Preferably the aromatic carboxylic acid used is anisic acid, in the form of alkali or alkaline-earth metal salts, ammonium salts or salts with an organic amine, or one of its C₁₋₄ alkyl esters.

The anisic acid used in the present invention may be used as it is or may be present in the form of an alkali or alkaline-earth metal salt, an ammonium salt or a salt with an organic amine, or in the form of a C₁₋₄ alkyl ester. The alkyl group of the ester may be linear or branched and there may be mentioned, by way of examples, methyl, ethyl, n-propyl, isopropyl, n-butyl and tert-butyl groups.

By way of examples of anisic acid salt or ester, there may be mentioned in particular sodium anisate, potassium anisate, methyl anisate, ethyl anisate, propyl anisate or butyl anisate.

In at least one embodiment, the at least one aromatic carboxylic acid is p-anisic acid or para-anisic acid.

The at least one aromatic carboxylic acid, or salt thereof is generally present in the composition of the present invention in an amount ranging from about 0.01% to 1% by weight; such as from about 0.05% to 0.5% by weight; such as from about 0.1% to 0.2% by weight, based on the total weight of the composition.

### Aromatic Alcohol

The sunscreen composition also contains about 0.01% to about 1% by weight of at least one aromatic alcohol. As used herein, the term “aromatic alcohol” means any compound that is liquid at room temperature and atmospheric pressure, comprising at least one entity chosen from benzene and naphthalene rings and at least one alcohol function (OH) directly linked to the ring or linked to at least one substituent of the ring. In at least one embodiment, the alcohol function may be on a substituent of the benzene or naphthalene ring.

Examples of aromatic alcohols that may be used in the composition include, but are not limited to: benzyl alcohol, benzoylisopropanol, benzyl glycol, phenoxyethanol, dichlorobenzyl alcohol, methylphenylbutanol, phenoxyisopropanol, phenylisohexanol, phenylpropanol, phenylethyl alcohol, and mixtures thereof.

Preferably the aromatic alcohol is phenoxyethanol.

The at least one aromatic alcohol is generally present in the composition of the present invention in an amount ranging from about 0.1% to 1% by weight; such as from about 0.2% to 0.8% by weight; such as from about 0.3% to 0.7% by weight, based on the total weight of the composition.

Further, preferably the combined weight percent of the aromatic carboxylic acid and aromatic alcohol, together forming the preservative system, is less than 1.7%, based on the total weight of the composition; preferably less than 1.3%; more preferably less than 1%.

Moreover, preferably the combined weight percent of the aromatic carboxylic acid, aromatic alcohol and any additional preservative or any material known to have anti-microbial activity in the composition is less than about 2.2%.

### Sunscreen Active

The sunscreen composition also contains at least one sunscreen active.

Preferably the sunscreen active is an organic sunscreen active.

Examples of organic sunscreen active include, but are not limited to, butyl methoxydibenzoylmethane; anthranilates; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications. U.S. Pat. No. 5,237,071, U.S. Pat. No. 5,166,355, GB 2 303 549, DE 197 26 184 and EP 893 119; benzoxazole derivatives such as those described in patent applications EP 0 832 642; EP 1 027 883, EP 1 300 137 and DE 101 62 844; screening polymers and screening silicones such as those described especially in patent application WO 93/04665; dimers derived from α-alkylstyrene, such as those described in patent application DE 198 55 649; 4,4-diarylbutadienes such as those described in patent applications EP 0 967 200, DE 197 46 654, DE 197 55 649, EP-A-1 008 586, EP 1 133 980 and EP 133 981, and mixtures thereof.

As examples of organic photoprotective agents, mention may be made of those denoted hereinbelow under their INCI name:

Butyl methoxydibenzoylmethane sold by A&E Connock (Perfumery & Cosmetics) LTD., also known as Avobenzone and also sold under the names “Eusolex 9020” sold by Merck KGaA/EMD Chemicals Inc., “Neo Heliopan” sold by Symrise, “Parsol 1789” sold by DSM Nuritional Products and “Oristar ABZ” sold by Orient Stars LLC.

Para-Aminobenzoic Acid Derivatives:

PABA,

Ethyl PABA,

Ethyl dihydroxypropyl PABA,

Ethylhexyl dimethyl PABA sold in particular under the name “Escalol 507” by ISP,

Glyceryl PABA,

PEG-25 PABA sold under the name “Uvinul P25” by BASF.

Salicylic Derivatives:

Homosalate sold under the name “Eusolex HMS” by Rona/EM Industries,

Ethylhexyl salicylate sold under the name “Neo Heliopan OS” by Haarmann and Reimer,

Dipropylene glycol salicylate sold under the name “Dipsal” by Scher,

TEA salicylate sold under the name “Neo Heliopan TS” by Haarmann and Reimer.

β,β-Diphenylacrylate Derivatives:

Octocrylene sold in particular under the trade name “Uvinul N539” by BASF,

Etocrylene sold in particular under the trade name “Uvinul N35” by BASF.

Benzophenone Derivatives:

Benzophenone-1 sold under the trade name “Uvinul 400” by BASF,

Benzophenone-2 sold under the trade name “Uvinul D50” by BASF,

Benzophenone-3 or Oxybenzone sold under the trade name “Uvinul M40” by BASF,

Benzophenone-4 sold under the trade name “Uvinul MS40” by BASF,

Benzophenone-5,

Benzophenone-6 sold under the trade name “Helisorb 11” by Norquay,

Benzophenone-8 sold under the trade name “Spectra-Sorb UV-24” by American Cyanamid,

Benzophenone-9 sold under the trade name “Uvinul DS-49” by BASF,

Benzophenone-12

Diethylaminohydroxybenzoylhexyl benzoate sold under the trade name “Uvinul A Plus” by BASF,

Benzylidenecamphor Derivatives:

3-Benzylidenecamphor manufactured under the name “Mexoryl SD” by Chimex,

4-Methylbenzylidenecamphor sold under the name “Eusolex 6300” by Merck,

Benzylidenecamphorsulfonic acid manufactured under the name “Mexoryl SL” by Chimex,

Camphor benzalkonium methosulfate manufactured under the name “Mexoryl SO” by Chimex,

Terephthalylidenedicamphorsulfonic acid manufactured under the name “Mexoryl SX” by Chimex,

Polyacrylamidomethylbenzylidenecamphor manufactured under the name “Mexoryl SW” by Chimex.

Phenylbenzimidazole Derivatives:

Phenylbenzimidazolesulfonic acid sold in particular under the trade name “Eusolex 232” by Merck,

Disodium phenyl dibenzimidazole tetrasulfonate sold under the trade name “Neo Heliopan AP” by Haarmann and Reimer.

Phenylbenzotriazole Derivatives:

Drometrizole trisiloxane sold under the name “Silatrizole” by Rhodia Chimie,

Methylenebis(benzotriazolyl)tetramethylbutylphenol sold in solid form under the trade name “MIXXIM BB/100” by Fairmount Chemical, or in micronized form as an aqueous dispersion under the trade name “Tinosorb M” by Ciba Specialty Chemicals.

Triazine Derivatives:

Bis(ethylhexyloxyphenol)methoxyphenyl triazine sold under the trade name “Tinosorb S” by Ciba-Geigy,

Ethylhexyltriazone sold in particular under the trade name “Uvinul T150” by BASF,

Diethylhexylbutamidotriazone sold under the trade name “Uvasorb HEB” by Sigma 3V,

2,4,6-tris(diisobutyl 4′-aminobenzalmalonate)-s-triazine.

Anthranilic Derivatives:

Menthyl anthranilate sold under the trade name “Neo Heliopan MA” by Haarmann and Reimer.

Imidazoline Derivatives:

Ethylhexyldimethoxybenzylidenedioxoimidazoline propionate.

Benzalmalonate Derivatives:

Polyorganosiloxane containing benzalmalonate functions, for instance Polysilicone-15, sold under the trade name “Parsol SLX” by Hoffmann LaRoche.

4,4-Diarylbutadiene Derivatives:

1,1-Dicarboxy(2,2′-dimethylpropyl)-4,4-diphenyl-butadiene.

Benzoxazole Derivatives:

2,4-bis[5-(1-dimethylpropyl)benzoxazol-2-yl(4-phenyl)imino]-6-(2-ethylhexyl)imino-1,3,5-triazine sold under the name Uvasorb K2A by Sigma 3V

and mixtures thereof.

The sunscreen active is typically present in an amount of from about 1% to about 50% by weight, such as from about 2% to about 47% by weight, and from about 3% to about 45% by weight, based on the total weight of the composition.

### Cosmetically Acceptable Aqueous Carrier

The sunscreen composition is carried in a cosmetically acceptable medium that is non toxic and can be applied to the skin, lips, hair, scalp, lashes, brows, nails or any other cutaneous region of the body. The composition of the invention may especially constitute a cosmetic or dermatological composition. The subject composition can be provided in a plethora of forms, including but not limited to creams, liquid, gel, cream-gel, lotion, serum, oil, a powder or a solid tube, and may optionally be packaged as an aerosol and may be in the form of a mousse or a spray, prepared according to the usual methods.

The cosmetically acceptable aqueous carrier comprises water. It may, however, if desired, also comprise alcohol, organic solvents, hydrocarbons, esters, silicones, and mixtures thereof.

The compositions according to the invention may be prepared according to techniques that are well known to those skilled in the art, in particular those intended for the preparation of emulsions of oil-in-water, water-in-oil, silicone-in-water and/or water-in-silicone type. They may be in particular in the form of a simple or complex emulsion (O/W, W/O, O/W/O,W/O/W and/or W/Si emulsions) such as a cream or a milk, in the form of a gel or a cream-gel, oil, or in the form of a lotion, a powder or a solid tube, and may optionally be packaged as an aerosol and may be in the form of a mousse or a spray. These compositions are prepared according to the usual methods.

Particularly, the compositions according to the invention may be in the form of an oil-in-water or water-in-oil emulsion.

The emulsions generally contain at least one emulsifier chosen from amphoteric, anionic, cationic and nonionic emulsifiers, which are used alone or as a mixture. The emulsifiers are appropriately chosen according to the emulsion to, be obtained (W/O or O/W).

As emulsifying surfactants that may be used for the preparation of the W/O emulsions, examples that may be mentioned include sorbitan, glycerol or sugar alkyl esters or ethers; silicone surfactants, for instance dimethicone copolyols, such as the mixture of cyclomethicone and of dimethicone copolyol, sold under the name “DC 5225 C” by the company Dow Corning, and alkyldimethicone copolyols such as laurylmethicone copolyol sold under the name “Dow Corning 5200 Formulation Aid” by the company Dow Corning; cetyldimethicone copolyol, such as the product sold under the name Abil EM 90R by the company Goldschmidt, and the mixture of cetyldimethicone copolyol, of polyglyceryl isostearate (4 mol) and of hexyl laurate, sold under the name Abil WE 09 by the company Goldschmidt. One or more co-emulsifiers may also be added thereto, which may be chosen advantageously from the group comprising polyol alkyl esters'. Polyol alkyl esters that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, PEG-30 dipolyhydroxy stearate, such as the product sold under the name Arlacel 135 by the company ICI and mixtures thereof.

For the O/W emulsions, examples of emulsifiers that may be mentioned include nonionic emulsifiers such as oxyalkylenated (more particularly polyoxyethylenated) fatty acid esters of glycerol such as the mixture of glyceryl stearate and PEG-100 stearate sold, for example, by the company ICI under the trade name Arlacel 165; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate; fatty alkyl ethers of sugars, especially polyalkylglucosides (APG) such as decylglucoside and laurylglucoside sold, for example, by the company Henkel under the respective names Plantaren 2000 and Plantaren 1200, cetostearyl glucoside optionally as a mixture with cetostearyl alcohol, sold, for example, under the name Montanov 68 by the company SEPPIC, under the name Tegocare CG90 by the company Goldschmidt and under the name Emulgade KE3302 by the company Henkel, and also arachidyl glucoside, for example in the form of a mixture of arachidyl alcohol, behenyl alcohol and arachidyl glucoside, sold under the name Montanov 202 by the company SEPPIC. According to one particular embodiment of the invention, the mixture of the alkylpolyglucoside as defined above with the corresponding fatty alcohol may be in the form of a self-emulsifying composition as described, for example, in document WO-A-92/06778.

It should also be noted that in the event that the cosmetically acceptable carrier is a meta-stable emulsion, both water-in-oil and oil-in-water emulsifiers should be employed.

Oils that may be, mentioned include mineral oils (paraffin); plant oils (sweet almond oil, macadamia oil, grapeseed oil or jojoba oil); synthetic oils, for instance perhydrosqualene, fatty alcohols, fatty acids or fatty esters (for instance the C₁₂-C₁₅ alkyl benzoate sold under the trade name “Finsolv TN” of “Witconol TN” by the company Witco, octyl palmitate, isopropyl lanolate and triglycerides, including capric/caprylic acid triglycerides), oxyethylenated or oxypropylenated fatty esters and ethers; silicone oils (cyclomethicone and polydimethylsiloxanes, or PDMS) or fluoro oils, and polyalkylenes.

Among the organic solvents that may be mentioned are lower alcohols and polyols. These polyols may be chosen from glycols and glycol ethers, for instance ethylene glycol, propylene glycol, butylene glycol, dipropylene glycol or diethylene glycol.

In another embodiment of the invention, the subject compositions are formulated as water-in-silicone or silicone-in-water emulsions in which the continuous oily phase comprises at least one silicone oil. When the compositions of the invention are formulated as water-in-silicone emulsions, the silicone oils are preferably present in a proportion of at least 5 percent and preferably ranging from 10 percent to 45 percent by weight with respect to the total weight of the emulsion. The fatty phase of the water-in-oil emulsions according to the invention can additionally comprise one or more hydrocarbon-comprising oil(s) in a proportion preferably ranging up to 40 percent by weight with respect to the total weight of the fatty phase of the emulsion.

Oils that may be used in the W/Si or Si/W composition may include for example: silicone oils, for instance volatile or non-volatile polymethylsiloxanes (PDMS) with a linear or cyclic silicone chain, which are liquid or pasty at room temperature, especially cyclopolydimethylsiloxanes (cyclomethicones) such as cyclohexasiloxane; polydimethyl-siloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenyl-siloxanes, diphenyl dimethicones, diphenylmethyl-diphenyltrisiloxanes or 2-phenylethyl trimethylsiloxy silicates, and polymethylphenylsiloxanes; mixtures thereof. Particularly representative of such oils are volatile silicone oils, such as cyclomethicones, in order to impart greater fastness towards water of the coloration on the skin, easier and more homogeneous spreading of the composition and shorter drying times.

For the W/Si emulsions, examples of emulsifiers generally include polyether-modified silicones having a long chain of dimethyl siloxane units which carry polyethoxy-polypropoxy units in the chain and at the ends. Examples include cyclopentasiloxane PEG/PPG-18/18 dimethicone, PEG-12 Dimethicone, and PEG/PPG-19/19 Dimethicone sold by Dow Corning under the name Dow Corning® BY 11-030.

It has also been surprisingly and unexpectedly discovered that an even more efficacious preservative system can be formulated by the further use of caprylyl glycol as a co-preservative.

The caprylyl glycol may be employed in an amount of from about 0.1 to about 2.0% by weight, preferably from about 0.1 to about 1.0% by weight, and most preferably from about 0.1 to about 0.5% by weight, all weights based on the total weight of the composition.

### Optional Ingredients

The composition may comprise at least one auxiliary ingredient, including for example active ingredients, film forming agents, surfactants, conditioning agents, adjuvents, self-tanners, colorants, skin active agents and mixtures thereof.

Among the active agents that may be mentioned are:

antipollution agents and/or free-radical scavengers;

depigmenting agents and/or propigmenting agents;

antiglycation agents;

NO-synthase inhibitors;

agents for stimulating the synthesis of dermal or epidermal macromolecules and/or for preventing their degradation;

agents for stimulating fibroblast proliferation;

agents for stimulating keratinocyte proliferation;

muscle relaxants;

tensioning agents;

desquamating agents;

moisturizers;

anti-inflammatory agents;

agents acting on the energy metabolism of cells;

insect repellants;

substance P or CGRP antagonists.

The compositions, in an embodiment of the present invention, can employ at least one aesthetic modifier. Aesthetic modifiers can be used to enhance the texture of the composition. For example, the compositions of the current invention can have a matte, powdery, non greasy, soft texture. Aesthetic modifiers can be chosen from: Nylon-12, polymethylsilsesquioxane, styrene/acrylates copolymer, silica and/or mixtures thereof.

The composition may also comprise standard cosmetic adjuvants chosen especially from fatty substances, organic solvents, ionic or nonionic, hydrophilic or lipophilic thickeners, softeners, humectants, opacifiers, stabilizers, emollients, silicones, antifoams, fragrances, preserving agents, anionic, cationic, nonionic, zwitterionic or amphoteric surfactants, active agents, fillers, propellants, acidifying or basifying agents, co-emulsifiers or any other ingredient usually used in cosmetics and/or dermatology.

The pH of the composition will generally range from about 4 to about 6.7. In at least one embodiment, the pH may range from about 4.5 to about 6, and may be adjusted to the desired value by means of acidifying or basifying agents that are well known to a person skilled in the art.

The composition of the present invention may also contain at least one tactile modifier. Suitable tactile modifiers include, but are not limited to nylon, silica, and mixtures thereof.

The composition of the present invention may also contain at least one colorant. Preferably, such colored compositions can be cosmetic compositions such as, for example, lip compositions (for example, lipstick or liquid lip colors or lip gloss), body tints, bronzers, self tanners, mascaras, eyeshadow, nail polish or foundations. According to this embodiment, the at least one colorant is preferably chosen from pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present the pigment is generally present in the composition of the present invention in an amount ranging from about 0% to about 15% by weight; such as from about 0.01% to about 10% by weight; such as from about 0.1% to about 5% by weight, all weights being based on the weight of the composition as a whole.

The composition of the present invention may contain emollients. Suitable emollients include, but are not limited to, mineral oil; petrolatum; C₇-C₄₀ branched chain hydrocarbons; C₁-C₃₀ alcohol esters of C₁-C₃₀ carboxylic acids; C₁-C₃₀ alcohol esters of C₂-C₃₀ dicarboxylic acids; monoglycerides of C₁-C₃₀ carboxylic acids; diglycerides of C₁-C₃₀ carboxylic acids; triglycerides of C₁-C₃₀ carboxylic acids; ethylene glycol monoesters of C₁-C₃₀ carboxylic acids; ethylene glycol diesters Of C₁-C₃₀ carboxylic acids; propylene glycol monoesters Of C₁-C₃₀ carboxylic acids; propylene glycol diesters of C₁-C₃₀ carboxylic acids; C₁-C₃₀ carboxylic acid monoesters and polyesters of sugars, for example, sefa cottonate (sucrose polycottonseedate); polydialkylsiloxanes; polydiarylsiloxanes; polyalkarylsiloxanes; cyclomethicones having 3 to 9 silicon atoms; vegetable oils; hydrogenated vegetable oils; polypropylene glycol C₄-C₂₀ alkyl ethers; di C₈-C₃₀ alkyl ethers; and mixtures thereof.

The composition of the present invention may also contain a self-tanning active agent. Suitable self tanning agents include, but are not limited to, monocarbonyl or polycarbonyl compounds, such as isatin, alloxan, ninhydrin, glyceraldehyde, mesotartaric aldehyde, glutaraldehyde, erythrulose and dihydroxyacetone (DHA).

The composition of the present invention may contain a skin lightening/whitening agent. Suitable skin lightening/whitening agents include, but are not limited to, hydroquinone; azelaic acid and other related dicarboxylic acids, and salts and derivatives thereof; retinoids; kojic acid; arbutin; nicotinic acid and its precursors, salts and derivatives; arbutin; ascorbic acid and salts and derivatives thereof (e.g., magnesium ascorbyl phosphate or sodium ascorbyl phosphate); herbal extracts (e.g., licorice extract, mulberry extract); and mixtures thereof.

Hydrophilic thickeners that may be mentioned include carboxyvinyl polymers such as the Carbopol products (carbomers) and the Pemulen products (acrylate/C10-C30-alkylacrylate copolymer); polyacrylamides, for instance the crosslinked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, which are optionally crosslinked and/or neutralized, for instance the poly(2-acrylamido-2-methylpropanesulfonic acid) sold by the company Hoechst under the trade name “Hostacerin AMPS” (CTFA name: ammonium polyacryldimethyltauramide); cellulose-based derivatives such as hydroxyethylcellulose; polysaccharides and especially gums such as xanthan gum; and mixtures thereof.

Needless to say, a person skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

The present invention will be better understood from the examples that follow, all of which are intended for illustrative purposes only and are not meant to unduly limit the scope of the invention in any way.

### EXAMPLES

The present invention is further described in terms of the following non-limiting examples. Unless otherwise indicated, all parts and percentages are on a weight-by-weight percentage basis.

| | Example 0 | Example 1 | Example 2 |
|---|---|---|---|
| chelating agent | 0.1 | 0.1 | 0.1 |
| solvent | 9.6 | 9.6 | 8.8 |
| Emulsifier stabilizer | 1.2 | 1.2 | 0.9 |
| neutralizer | 0.02 | 0.02 | 0.02 |
| p-ANISIC ACID | 0.0 | 0.1 | 0.1 |
| emollient | 4 | 4 | 4 |
| emulsifier | 0.75 | 0.75 | 0.75 |
| aesthetic Modifier | 2.7 | 2.7 | 2.7 |
| thickener | 1.15 | 1.15 | 1.15 |
| SPF Booster | 3 | 3 | 3 |
| wax | 1 | 1 | |
| PHENOXYETHANOL | 0.5 | 0.7 | 0.5 |
| Caprylyl Glycol | | | 0.5 |
| METHYLPARABEN | 0.25 | | |
| WATER | QS | QS | QS |
| antioxidant | 0.05 | 0.05 | 0.05 |
| UV filters | 34 | 34 | 34 |

Comparative Example 0 contained 0.5% wt. of aromatic alcohol via phenoxyethanol and 0.25% wt. of methylparaben as preservatives, and no aromatic carboxylic acid, or salt thereof;

Inventive Example 1 contained 0.7% wt. aromatic alcohol via phenoxyethanol, 0.1% aromatic carboxylic acid, or salt thereof, via p-anisic acid as the only preservatives;

Inventive Example 2 contained 0.5% wt. aromatic alcohol via phenoyethanol, 0.1% wt. aromatic carboxylic acid, or salt thereof, via p-anisic acid and 0.5% wt. of co-preservative caprylyl glycol.

Examples 0 to 2 underwent microbiological study per the following protocol:
1. The product was inoculated with saline suspensions made out of 24 hour culture of bacteria and yeast isolates and 5 days culture of mold isolate utilizing the following microorganisms:
   1.1 Pseudomonas aeruginosa 19429
   1.2 Escherichia coli 8739
   1.3 Staphylococcus aureus 6538
   1.4 Enterococcus faecalis 33186
   1.5 Candida albicans 10231
   1.6 Aspergillus niger 6275;
2. Initial concentration of microorganisms in the suspension was approximately 1.0E+8;
3. Inoculation rate was 1%;
4. Final concentration of microorganisms in the product was approximately 1.0E+6;
5. Number of surviving microorganisms was monitored at 7, 14 and 28 days after inoculation by the aerobic plate count method;
6. Logarithmic reduction of microorganisms was calculated from the theoretical initial concentration of microorganism in the product.

As can be seen from the tables below, the microbiological study results evidence the equivalence, in terms of preservative efficacy, with respect to Comparative Example 0 containing methylparaben and Inventive Example 1 containing the combination of phenoxyethanol and p-anisic acid. Example 2 which contained, as a co-preservative, caprylyl glycol, showed the most efficacious preservative results with less than 200 CFU/g (TOM/TA) at 7 days.

### Example 0

| | |
|---|---|
| | (TOM/TA) |
| Inoculum | CFU/g |

| MICROORGANIMS | CFU/g | 2 days | 7 days |
|---|---|---|---|
| Escherichia coli | 1.5E6 | | <200 |
| Enterococcus faecalis | 1.3E6 | | 6.0E5 |
| Candida albicans | 1.1E6 | | <200 |
| Aspergilus niger | 1.8E6 | | <200 |

### Example 1

| | |
|---|---|
| | (TOM/TA) |
| Inoculum | CFU/g |

| MICROORGANIMS | CFU/g | 2 days | 7 days |
|---|---|---|---|
| Escherichia coli | 1.5E6 | | <200 |
| Enterococcus faecalis | 1.3E6 | | 6.0E5 |
| Candida albicans | 1.1E6 | | <200 |
| Aspergilus niger | 1.8E6 | | <200 |

### Example 2

| | |
|---|---|
| | (TOM/TA) |
| Inoculum | CFU/g |

| MICROORGANIMS | CFU/g | 2 days | 7 days |
|---|---|---|---|
| Escherichia coli | 2.9E6 | <200 | <200 |
| Enterococcus faecalis | 1.9E6 | <200 | <200 |
| Candida albicans | 2.8E6 | <200 | <200 |
| Aspergilus niger | 1.5E6 | <200 | <200 |

What is claimed is:

1. A sunscreen composition comprising:
(a) at least one aromatic carboxylic acid, or salt thereof;
(b) from about 0.01% to about 1% by weight of at least one aromatic alcohol;
(c) at least one sunscreen active; and
(d) a cosmetically acceptable aqueous carrier.

2. The composition of claim 1, wherein (a) is chosen from benzoic acid, para-anisic acid, anisic acid, caffeic acid, chlorogenic acid, diphenolic acid, ferulic acid, hippuric acid, hydroxycinnamic acid, phenylthioglycolic acid, salicylic acid, acetylsalicylic acid, phthalic acid, salified forms thereof, and combinations thereof.

3. The composition of claim 1, wherein (a) is p-anisic acid.

4. The composition of claim 1, wherein (a) is present in an amount of from about 0.01% to about 1% by weight, based on the total weight of the composition.

5. The composition of claim 1, wherein (a) is present in an amount of from about 0.1% to about 0.2% by weight, based on the total weight of the composition.

6. The composition of claim 1, wherein (b) is chosen from benzyl alcohol, benzoylisopropanol, benzyl glycol, phenoxyethanol, dichlorobenzyl alcohol, methylphenylbutanol, phenoxyisopropanol, phenylisohexanol, phenylpropanol, phenylethyl alcohol, and combinations thereof.

7. The composition of claim 1, wherein (b) is phenoxyethanol.

8. The composition of claim 1, wherein (b) is present in an amount of from about 0.1% to about 1% by weight, based on the total weight of the composition.

9. The composition of claim 1, wherein (b) is present in an amount of from about 0.3% to about 0.7% by weight, based on the total weight of the composition.

10. The composition of claim 1, wherein the combined weight percent of (a) and (b) is less than 1.7% by weight, based on the total weight of the composition.

11. The composition of claim 1, wherein the combined weight percent of (a) and (b) and any additional preservatives in the composition is less than about 2.2%, based on the total weight of the composition.

12. The composition of claim 1, wherein (c) is an organic sunscreen active.

13. The composition of claim 1, wherein (c) is present in an amount of from about 1 to about 50% by weight, based on the total weight of the composition.

14. The composition of claim 1, wherein (c) is present in an amount of from about 3 to about 45% by weight, based on the total weight of the composition.

15. The composition of claim 1 having a pH ranging from about 4 to about 6.7.

16. The composition of claim 1, wherein (d) is chosen from water, alcohol, organic solvents, hydrocarbons, esters, silicones, and mixtures thereof.

17. The composition of claim 1 comprising at least one auxiliary ingredient chosen from film forming agents, surfactants, conditioning agents, adjuvents, self-tanners, colorants, skin active agents and mixtures thereof.

18. The composition of claim 1 further comprising caprylyl glycol.

19. The composition of claim 18 wherein the caprylyl glycol is present in an amount of from about 0.1 to about 2.0% by weight, based on the weight of the composition.

20. The composition of claim 18 wherein the caprylyl glycol is present in an amount of from about 0.1 to about 1.0% by weight, based on the weight of the composition.

21. The composition of claim 1 wherein the composition does not require parabens as a preservative.

22. A sunscreen composition comprising:
(a) from about 0.01% to about 1% by weight of at least one aromatic acid comprising an anisic acid, in the form of alkali or alkaline-earth metal salts, ammonium salts or salts with an organic amine, or one of its C₁₋₄ alkyl esters;
(b) from about 0.1% to about 1% by weight of at least one aromatic alcohol comprising phenoxyethanol;
(c) from about 3% to about 45% by weight of at least one sunscreen active;
(d) from about 0.1% to about 0.5% of caprylyl glycol; and
(e) remainder by weight to 100%, a cosmetically acceptable aqueous carrier,
wherein the weights of (a)-(d) are based on the total weight of the composition;
wherein the total amount of (a) and (b) is less than about 1.7; and
wherein the composition has a pH ranging from about 4.5 to about 6.

23. The composition of claim 22 wherein the composition does not require parabens as a preservative.

24. A method for protecting a keratinous substrate from harmful UV rays comprising topically applying a sunscreen composition containing:
(a) at least one aromatic carboxylic acid, or salt thereof;
(b) from about 0.1% to about 1% by weight of at least one at least one aromatic alcohol;
(c) at least one sunscreen active; and
(d) a cosmetically acceptable aqueous carrier.

25. The method of claim 24 wherein the composition further comprises caprylyl glycol.

26. The method of claim 24 wherein the composition does not require parabens.
